(19) United States
(12) Patent Application Publication
(10) Pub. No.: US 2017/0343566 A1
(43) Pub. Date: Nov. 30, 2017

(54) Saliva Collection Kit and Procedure for Validated Home Assessment of Dim Light Melatonin Onset

(71) Applicant: Rush University Medical Center, Chicago, IL (US)

(72) Inventor: Helen J. Burgess, Chicago, IL (US)

(73) Assignee: Rush University Medical Center, Chicago, IL (US)

(21) Appl. No.: 15/601,190

(22) Filed: May 22, 2017

Related U.S. Application Data
(60) Provisional application No. 62/342,669, filed on May 27, 2016.

(51) Int. Cl. G01N 33/74; A61B 5/00; A61B 10/00

(52) U.S. Cl. CPC G01N 33/74; A61B 10/0096; A61B 5/0059; A61B 10/0051; G01N 2800/2864

(57) ABSTRACT

Methods and kits are provided for collecting samples for assessment of circadian timing in a non-clinical environment. The methods include monitoring light exposure of a subject and placing a first biological sample in a first sample vial, wherein the first biological sample is obtained in the non-clinical environment in dim light comprising light less than about 50 lux and monitoring the use of the first sample vial with a monitoring device. The methods include placing a second biological sample from the subject in a second sample vial, the second biological sample being taking at a time interval after the first biological sample and monitoring the use of the second sample vial with the monitoring device. Kits include a photosensor, a plurality of sample vials free from labels, a label system for labeling the plurality of sample vials, and a monitoring device for monitoring use of the plurality of sample vials.

### RELATED APPLICATIONS

This application claims the benefit of U.S. Provisional Patent Application No. 62/342,669, filed on May 27, 2016, which is incorporated herein by reference in its entirety.

### FEDERALLY SPONSORED RESEARCH OR DEVELOPMENT

This invention was made with government support under grant number R01 AT007104 awarded by the National Center for Complementary and Alternative Medicine. The government has certain rights in the invention.

### BACKGROUND

### 1. Technical Field

Methods and kits for collecting samples for assessment of circadian timing. The methods and kits facilitate collection of samples in a non-clinical environment.

### 2. Background Information

The most reliable measure of central circadian timing in humans is the onset of melatonin secretion, when measured in dim light conditions (dim light melatonin onset, DLMO).^{1,2} Melatonin typically begins to rise in the 2-3 h before the usual onset of nocturnal sleep,³ but must be measured in dim light because light can suppress melatonin secretion.⁴ The measurement of the DLMO is now encouraged in the latest diagnostic criteria for circadian rhythm sleep disorders (International Classification of Sleep Disorders, Third Edition).⁵ Additionally, measuring the DLMO can help to optimize the treatment of circadian rhythm sleep disorders with melatonin or bright light⁶⁻⁸ and help to prevent patients from receiving treatment at the wrong circadian time, which risks worsening their condition.^{7,9} Similarly, measuring the DLMO or “phase typing” patients with winter depression can assist in optimizing the timing of bright light treatment.¹⁰

The DLMO is most frequently assessed in a research laboratory or clinic. Research participants or clinical patients are required to arrive at the facility approximately 6-8 h before their usual bedtime, and are guided by staff to remain in dim light, and to give samples every half hour or hour until their usual bedtime or even later.^{1,3} Melatonin can be measured in plasma, but melatonin is most easily assessed noninvasively from saliva samples.¹¹ Additionally, saliva is often sampled more frequently than the urinary melatonin metabolite 6-sulphatoxymelatonin, allowing for greater precision in measurement.¹¹ The need for staff and space considerably increases the expense and inconvenience associated with measuring the DLMO.-¹² Furthermore, some participants and/or patients are reluctant to stay late or overnight in an unfamiliar laboratory or clinic. Thus, the possibility of having research participants or patients collect saliva samples in their own homes instead of having to stay in the laboratory or clinic at night is very appealing.

However, other concerns arise when saliva sampling occurs at home and is not supervised by staff. The first is the need to ensure people are in sufficiently dim light to avoid melatonin suppression, and subsequent circadian phase shifting. To date, only one study has compared DLMOs generated from home saliva samples to DLMOs generated from saliva samples collected in the laboratory.¹² In this study, light exposure at home was not measured, but the authors estimated that approximately 20% of the home DLMOs were suppressed by light, as these home DLMOs occurred more than 1 h later in time than the corresponding laboratory DLMOs. A second concern surrounding home saliva sampling is sample timing. For example, in one study of home saliva sampling for later determination of cortisol levels, compliance to scheduled sample times was poor, especially when participants were not informed that they were being electronically monitored.¹³ On average, participants who were unaware they were being monitored gave saliva samples more than 2 h from the scheduled sample times, but nevertheless reported significantly better compliance to the study investigators.¹³ The authors concluded that “researchers cannot rely on participants' self-reports of sampling times”¹³.

What is needed in the art and in response to the increasing need for accurate home DLMOs, a kit and methods have been developed that are designed to facilitate home saliva sampling while including objective measures of compliance to the requirement for dim light levels and scheduled times for saliva samples. After collection, examination of the light levels and sample times can assist in determining whether the home procedures were correctly followed. Light exposure is measured in 30-sec epochs by a photosensor worn around the neck on a cord and pinned to the outermost clothing. This placement of the photosensor reduces the risk of sleeves covering a wrist-worn photosensor. Sample times are recorded by use of a medication monitoring device that tracks the opening of a vial that contains cotton swabs used for generating a saliva sample. The kit also includes a dispenser with prepared labels in chronological order, so the subject only has to attach a label and is not required either to select a specific tube, or to write the correct time on the tube, both of which can lead to errors in sample coding.^{12,14} To our knowledge this is the first kit for home saliva sampling that includes objective markers of light exposure and saliva sample timing, and a system to reduce sample labeling errors.

The kits and methods described herein may be used for home DLMO sample collection and may be used for assessing circadian timing and disorders associated with circadian timing.

### BRIEF SUMMARY

Methods of collecting samples for assessment of circadian timing in a non-clinical environment are provided. The methods include monitoring light exposure of a subject in a non-clinical environment. The methods also include placing a first biological sample from a subject in a first sample vial, wherein the first biological sample is obtained in the non-clinical environment in dim light comprising light less than about 50 lux and monitoring the use of the first sample vial with a monitoring device. The methods further include placing a second biological sample from the subject in a second sample vial, the second biological sample being taking at a time interval after the first biological sample and monitoring the use of the second sample vial with the monitoring device.

Kits for collecting samples for assessment of circadian timing are provided. Kits include a photosensor, a plurality of sample vials free from labels, a label system for labeling the plurality of sample vials, and a monitoring device for monitoring use of the plurality of sample vials.

### BRIEF DESCRIPTION OF THE DRAWINGS

FIG. 1 illustrates sample protocols for a subject who typically slept from 23:00 to 07:00. Participants were randomized to Protocol A or Protocol B. Protocol A consisted of a home phase assessment, a laboratory phase assessment, a 5-day break, a laboratory phase assessment, and a home phase assessment. Protocol B consisted of a laboratory phase assessment, a home phase assessment, a 5-day break, a home phase assessment and a laboratory phase assessment. The gray rectangles represent the time required for dime light. The dot represents the time of the first saliva sample with saliva sampling continuing every 30 min up until 2 h after average bedtime. The black rectangles represent scheduled sleep times. Square brackets indicate approximate arrival and departure times from the laboratory.

FIG. 2 illustrates the clock time of dim light melatonin onsets (DLMOs) collected in laboratory phase assessments versus home phase assessments. The two measures were highly correlated (r=0.91, P<0.001). The line is the line of unity.

FIG. 3 illustrates Individual melatonin profiles collected in a home phase assessment either the day before or day after a laboratory phase assessment. Top panel: An example of when the home dim light melatonin onset (DLMO) occurred before the laboratory DLMO. Middle panel: An Example of when the home DLMO occurred at the same time as the laboratory DLMO. Bottom panel: An example of when the home DLMO occurred after the laboratory DLMO.

FIG. 4 illustrates the distribution of the difference between the laboratory dim light melatonin onsets (DLMOs) and home DLMOs, calculated by subtracting each home DLMO from its corresponding laboratory DLMO. The zero line represents no difference between the DLMOs, a positive difference reflects the home DLMO occurring earlier in time than the corresponding laboratory DLMO, while a negative difference reflects the home DLMO occurring after the corresponding laboratory DLMO. The solid lines represent the mean differences in each protocol. The dashed lines represent a 30-min difference and a 1 h difference between the home and laboratory DLMOs.

FIG. 5 illustrates sample protocols for a delayed sleep phase disorder (DSPD) participant who slept typically from 02:00 to 10:00 hours. Participants were randomized to protocol A or B. Grey rectangles represent dim light; dots represent first saliva sample; black rectangles represent scheduled sleep times. Square brackets encompass times in the laboratory.

FIG. 6 illustrates clock time of dim light melatonin onsets (DLMOs) collected from delayed sleep phase disorder participants (DSPDs) in laboratory versus home assessments. The lines of unity±1 h are shown.

FIG. 7 illustrates individual melatonin profiles collected at home before or after a laboratory assessment. Top: home dim light melatonin onset (DLMO) before laboratory DLMO; middle: home DLMO within 30 min of laboratory DLMO; bottom: home DLMO after laboratory DLMO.

FIG. 8 illustrates the difference between the laboratory and home dim light melatonin onsets (DLMO). Open circles represent when the home DLMO was collected first. The solid lines represent the mean differences, dashed lines represent 30-min and 1-h differences.

FIG. 9 illustrates an embodiment of a kit that may be used to collect the saliva samples for the DLMO monitoring.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

The present invention relates to methods and kits for collecting samples for assessment of circadian timing. The methods and kits facilitate collection of samples in a non-clinical environment. By way of non-limiting example, the sample collection may need to be collected in a dim light setting to assess the circadian timing. In some aspects, the sample may be a saliva sample. In some aspects, monitoring the timing of the sample collection is needed and multiple samples are collected over a time period. The methods and kits are designed to minimize sampling errors in a non-clinical environment. By way of non-limiting example, sampling errors may include exposure of the subject to light during the sample collection time period and/or labeling errors on the sample vials and/or timing of the sample collection.

As used herein, the term “non-clinical environment” refers to a setting that is not laboratory, such as a sleep laboratory, or a clinic or a hospital where a clinician would typically be responsible for monitoring light, time and sample labeling. In some embodiments, the non-clinical environment is the subject's home. The home environment may be beneficial for subjects that do not wish to spend time in a clinical environment or who cannot travel from home or be away for the length of the sample collection. The home environment may also be beneficial for reducing costs of the sample collection by reducing such factors such as personnel and space costs associated with a clinical environment.

The terms “sample” or “biological sample” as used herein, refers to a sample of biological fluid, tissue, or cells, in a healthy and/or pathological state obtained from a subject. The sample may be, for example, saliva, blood, urine, lachrymal fluid, plasma, or serum. In some embodiments, the sample is a saliva sample.

The term “subject” or “patient” as used herein, refers to a mammal, preferably a human.

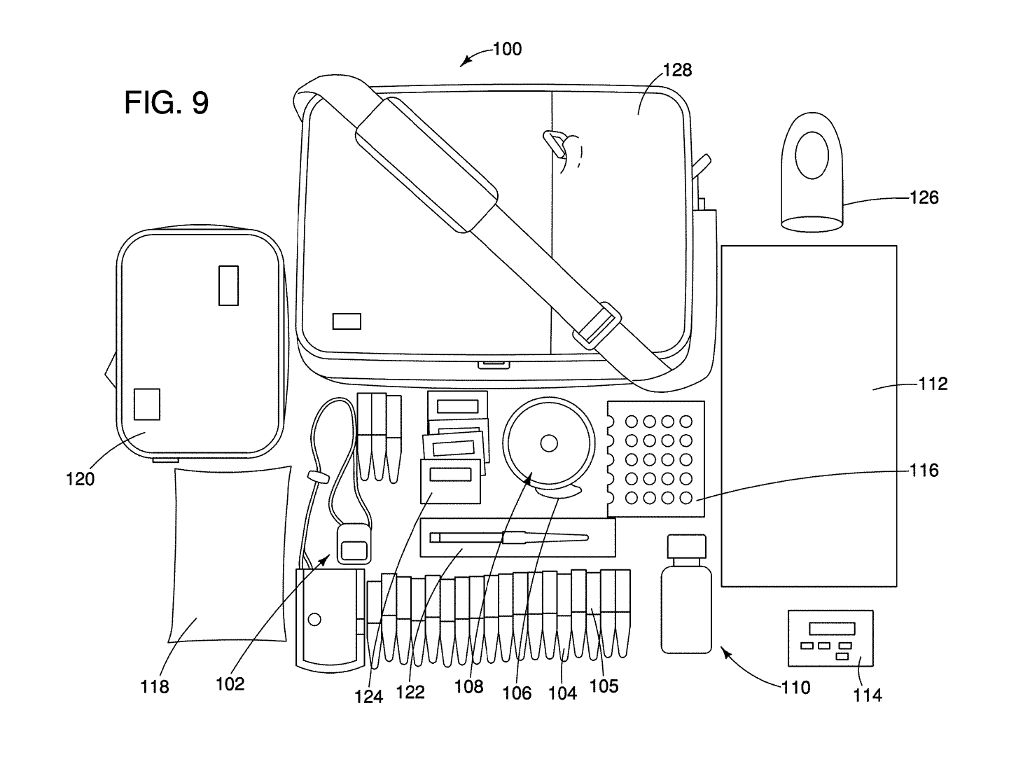

Kits may be provided to the subjects for use in sample collection in a non-clinical environment. The kits may include one or more components for facilitating collection of the samples. An example kit 100 is shown in FIG. 9. As shown, the kit 100 may include a photosensor 102 for monitoring the amount of light the subject is exposed to during the sample collection. (For example, Actiwatch Spectrum, Respironics, Bend, Oreg.) In some embodiments, the photosensor 102 may be configured to measure light exposure in 30-sec epochs, although the time may be longer or shorter. The photosensor 102 may be worn on an outer layer of the subject's clothing so that photosensor 102 is free from covering by the subject's clothing. In some embodiments, the photosensor 102 may be worn on the subject's wrist provided that the photosensor 102 remains free from covering during the monitoring period.

The kit may include a plurality of sample vials 104 for facilitating collection of the samples during the monitoring period. For each vial 104, a swab 105 may be included, for example for placement into the sample vial when saliva samples are collected. By way of non-limiting example, the sample vials 104 for the kit 100 may be purchased (e.g. Salivettes, Sarstedt, Newton, N.C.) or may be assembled using tubes with swabs 105 provided separately. The number of sample vials 104 provided in the kit 100 may be equal to the number of samples to be collected during the monitoring period. In some embodiments, extra sample vials 104, greater than the number of samples may be provided. The sample vials 104 are provided in the kit 100 free of labels so that a pre-coded label 106 provided separately with the kit 100 may be added to the sample vial 104 during the sample collection. In some embodiments, the pre-coded labels 106 are provided in a label dispenser 108 so that the subject removes the pre-coded labels 106 in chronological order for placement on the sample vials 104. The pre-coded labels 106 help to minimize collection errors so that the subject does not need to select a specific tube or label the tube with a specific time.

The kit may include a monitoring device 110 to monitor the timing of the collection of the plurality of samples in the sample vials 104 for collected from the subject during the monitoring period. By way of non-limiting example, the cotton swabs 105 may be provided in the kit 100 within the monitoring device 110. For example, the monitoring device 110 may be a TrackCap device that includes a chip in the lid of the cap that records the opening and/or closing times of the lid so that the sample collection time is recorded for each sample. (AARDEX, Union City, Calif.). At the appropriate time, the subject removes one swab 105 from the monitoring device 110 so that the time is recorded by the monitoring device when the swab 105 is removed. After the sample is collected, the swab 105 is placed in the sample vial 104 and the pre-coded label 106 is added.

The kit 100 may also include a check list of instructions 112 that may include sample times and a timer 114. The timer 114 may be programmed with times for the sample collection during the monitoring period. (PalmOne Tungsten E, Handheld, Hewlett Packer, Palo Alto, Calif.) In some embodiments, the timer 114 may include audio instructions. By way of non-limiting example, the timer may be any device that tracks time and signals when a time point is reached, such as an alarm clock, a watch, a timer, a phone and the like.

In some embodiments, the kit 100 may include a foam rack 116 for storage of the sample vials 104, an ice pack 118, and a case 120 for transporting the sample vials 104 to the laboratory for measurement of the samples after the monitoring period is completed. Some kits 100 may also include a toothbrush 122, a pain relief medication 124 that will not interfere with the sample testing, a night light 126 that emits dim light less than about 50 lux, and a case 128 for transporting the kit 100.

Methods are also provided collecting samples for assessment of circadian timing. Subjects may use the kits 100 described above for collecting samples at specified times for the duration of a monitoring period. The methods for assessing circadian timing include monitoring light exposure of a subject in a non-clinical environment. In some aspects, samples from the subject are collected from the subject in a dim light where the light exposure of the subject is less than about 50 lux. A photosensor may be worn by the subject to monitor the light exposure during the monitoring period. The photosenor is worn by the subject on the outermost layer of the subject's clothing.

In some embodiments, the subject collects a first sample in a first sample vial 104 at a first time point while the subject is in the dim light. The collection of the first sample is monitored using a monitoring device. At a second, later time point, the subject collects a second sample. The collection of the second sample is monitored using the monitoring device. The samples may be collected by the subject or by an assistant.

In some aspects, the monitoring period may begin about 30 min prior to the first sample collection time point where the subject is prompted by the timer to dim the lights, close the blinds and dim the screens of electronic devices in the non-clinical environment so that the light exposure to the subject is about 50 lux or less. The monitoring period may begin at any time sufficient to avoid interference with bright light exposure influencing the sample collection and later testing, for example when melatonin is measured. After the light exposure has been adjusted to less than about 50 lux for a sufficient period of time, the timer prompts the subject to collect samples at specific time intervals. The time intervals may be any time interval that monitors circadian timing. For example, the time interval may be about but is not limited to 15 min, 20 min, 30 min, 40 min, 45 min, 50 min, 60 min and intervals in between, or longer or shorter. Similarly, the monitoring period may be any time sufficient to monitor the circadian timing of the subject. For example, the monitoring period may be about 3 hours, 4 hours, 5 hours, 6 hours, 6,5 hours, 7 hours, 7.5 hours, 8 hours, 8.5 hours, 9 hours, 9.5 hours, 10 hours, intervals in between, or longer or shorter.

In some aspects, swabs for sample collection may be provided in the monitoring device in the kit as described above. When prompted by the timer at the appropriate time interval, the subject obtains a sample by opening the cap of the monitoring device and removing a swab, replacing the cap of the monitoring device and obtaining a sample. The opening and/or closing of the cap of the monitoring device provides a time stamp for the sample collection. The sample is collected, such as a saliva sample, and the swab is placed in a sample vial. A pre-coded label is added to the sample vial during the collection procedure. Sampling in dim light continues for the duration of the monitoring period. The samples may be frozen and taken to a lab to be measured after the monitoring period is completed.

### EXAMPLES

### Example 1

### Home Versus Laboratory DLMOs

Methods

Participants

Thirty-five healthy participants participated in the study year round (12 in spring, 4 in summer, 7 in fall, 12 in winter). All participants were medication free, consumed only moderate caffeine (<300 mg/day) and alcohol doses (<2 drinks/day), and had a body mass index between 18.5-29.8 kg/m². Based on their responses to screening questionnaires, all participants had no medical (Tasto Health Questionnaire¹⁵), psychiatric (Minnesota Multiphasic Personality Inventory-2,¹⁶ Beck Depression Inventory,¹⁷ Personal Inventory for Depression and Seasonal Affective Disorder¹⁸), or sleep disorders (Pittsburgh Sleep Quality Index,¹⁹Insomnia Severity Index,²⁰ Berlin Sleep Apnea Questionnaire,²¹ International Restless Legs Syndrome Study Group consensus criteria for restless leg syndrome²²) and were not extreme chronotypes (Owl and Lark Questionnaire²³). All participants were required to pass a urine drug screen for common drugs of abuse and nicotine; two participants failed the urine test and were dropped from the study. A third subject was dropped from the study after an urgent work assignment conflicted with study participation. The remaining sample of 32 participants consisted of 16 men, 16 women; 21-62 y, mean age±standard deviation 39.9±13.9 y. There were 11 moderate morning, 16 neither types, and five moderate evening types in the final sample. Almost all participants had some college education (94% of sample), with the remainder having only completed high school. Most participants were employed on a part-time (44% of sample) or full-time (31% of sample) basis, with the remainder reporting that they were not working. The majority of participants were not students (88% of sample), with only a minority reporting student status (12% of sample). No subject was color blind, as determined by the Ishihara test for color blindness. All participants had not worked any night shifts in the 2 y prior to the study and had not traveled across more than one time zone in the 2 mo preceding the study. All participants reported no previous experience with saliva sampling, and had not previously participated in any research study in our laboratory. Non-steroidal anti-inflammatory drugs were not permitted throughout the study because they can suppress melatonin. All participants gave written informed consent prior to their participation. The study was approved by the Rush University Medical Center Institutional Review Board.

Protocols

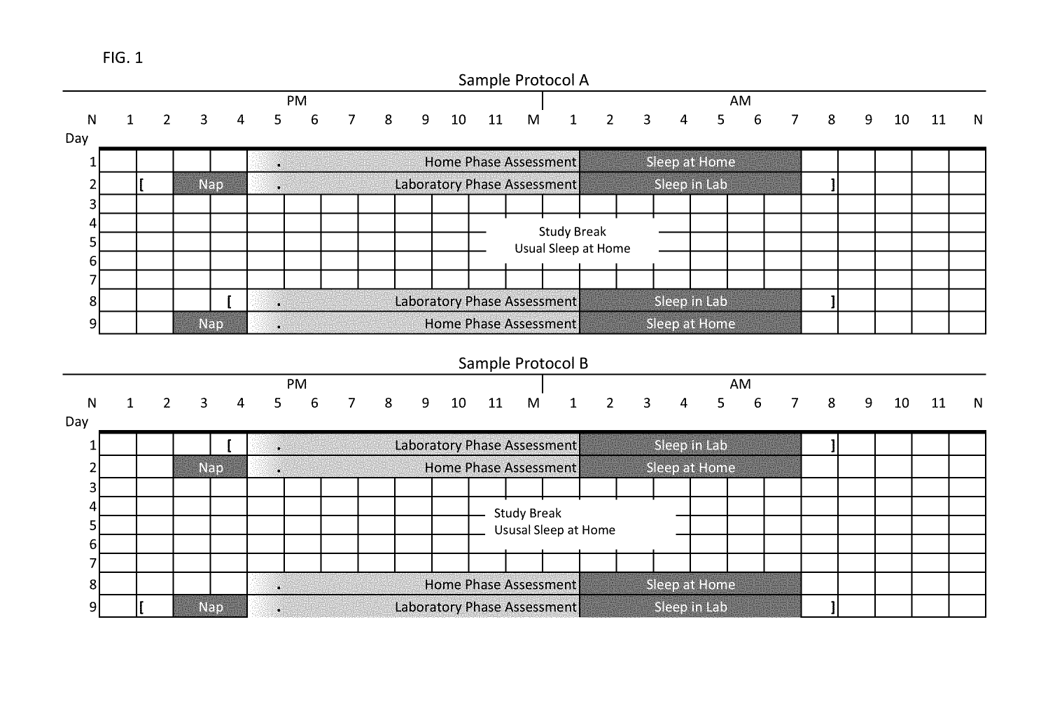

Participants were randomized in groups of one to three people to one of two 10-day protocols (FIG. 1). In Protocol A, participants completed a home circadian phase assessment first, followed by a laboratory circadian phase assessment the next day. Participants then had a 5-day break where they returned to their usual sleep schedule at home, before completing a laboratory phase assessment, followed the next day by a second home phase assessment. In Protocol B, participants completed a laboratory phase assessment first, followed by a home phase assessment the next day. Participants then had a 5-day break where they returned to their usual sleep schedule at home, before completing a home phase assessment, followed the next day by a laboratory phase assessment. Sixteen participants completed Protocol A and 16 participants completed Protocol B.

The protocol for each subject was tailored to each individual's habitual sleep times collected in the week before the study start with daily sleep diaries. Subjects were not required to follow a fixed sleep-wake schedule during this week. On average across the sample, each participant's bedtime varied by a maximum of 89.8 min and each participant's wake time varied by a maximum of 114.5 min during this week. Saliva sampling started 6 h before and ended 2 h after each subject's average bedtime (FIG. 1). After the last saliva sample, participants slept at home or in the laboratory before waking at their average wake time, to minimize any shifts in circadian timing. The average bedtime and wake time for the sample was 23:43±0.9 h and 07:41±0.7 h respectively, with average bedtime in the sample ranging from 21:30 to 01:00 and average wake time ranging from 06:30 to 09:00. Participants were required to take a 2-h nap prior to the second and fourth phase assessment (whether at home or in the laboratory) to reduce the sleep deprivation from the night before. Driving was not permitted on any study day where the protocol had led participants to be sleep deprived. All participants wore a wrist actigraphy monitor (30-sec epochs, Actiwatch Spectrum, Respironics, Bend, Oreg.) on their nondominant wrist throughout the 10-day study to ensure compliance to the study protocol.

Laboratory Circadian Phase Assessments

When in the laboratory participants were continuously supervised by research staff, and guided through the laboratory procedures. Participants were required to remained awake and seated in dim light (<5 lux, at level of the eyes, in direction of gaze, measured every 2 h, Extech 403125 light meter, Nashua, N.H.) starting 6.5 h before their average bedtime (FIG. 1). After 30 min in the dim light, participants were prompted by staff to give a saliva sample every 30 min using Salivettes (Sarstedt, Newton, N.C.). The participants tipped the cotton swab from the Salivette into their mouths, and rolled the cotton swab in their mouths for up to 5 min until saturated, before spitting it back into the Salivette. This procedure continued every 30 min until the last saliva sample, which occurred 2 h after their average bedtime. Toothpaste or mouthwash was not allowed during the phase assessments. Small snacks and fluids were permitted, except in the 10 min before each sample, and participants were required to rinse and brush their teeth with water while remaining seated 10 min before each sample if they had consumed food or drink. Participants remained seated throughout the laboratory phase assessment except for bathroom trips, but these were not permitted in the 10 min before each sample. Participants were not permitted to consume any alcohol or caffeine at least 24 h before each phase assessment and were breathalyzed on arrival at the laboratory.

Home Circadian Phase Assessments with Measures of Compliance

The home phase assessments were designed to be as similar as possible to the laboratory phase assessments, with the addition of objective markers of compliance to the requirement for dim light and correct sampling times. Participants met a staff member at the laboratory earlier in the day of each home phase assessment. During these appointments, participants received a “light medallion” (Actiwatch Spectrum, Respironics, Bend, Oreg., 30-sec epochs, with wrist band removed, strung onto a cord worn around the neck with an attached safety pin), were instructed on the home procedures, witnessed a demonstration of how to collect a saliva sample, and received a home saliva collection kit. As for the laboratory phase assessments, participants were not permitted to consume any alcohol or caffeine at least 24 h before each home phase assessment and were breathalyzed during their visit to the laboratory. Participants were advised of the need to prepare food ahead of time so they could snack in between the half hourly saliva samples at home. A return appointment was made for the next day, so that participants could return the home kit. Participants were informed that their compliance to the home procedures was being monitored and the data would be examined by staff in their presence during the return appointment. The time taken to explain the home kits and home procedure varied between 20 to 30 min, depending on questions from participants.

The home saliva collection kit consisted of the following: a timer (PalmOne Tungsten E Handheld, programmed with Palm Desktop 4.1.4 software, Hewlett Packard, Palo Alto, Calif.), a paper checklist, a foam test tube rack, a small insulated bag with removable ice pack, 17 Salivettes with the cotton swabs removed, a vial with a MEMS TrackCap lid (microchip time stamps each lid opening, MWV Healthcare, Richmond Va.) with the 17 cotton swabs inside, a dispenser with prepared labels in chronological order, a soft toothbrush, eight Tylenol pills to replace any nonsteroidal anti-inflammatory drugs participants may wish to take in case of headaches and an event log for participants to note any odd events during the home phase assessment. Participants were offered a night light to assist in dimming their home bathrooms and 24 participants (75%) reported using the night light. The kit also contained three spare Salivettes, each with a cotton swab inside in case of an error with a saliva sample. The home kit was packed into a black messenger bag for easy transport home.

Upon arrival at home, participants were instructed to follow the checklist whenever prompted by the preset alarms on the timer, and to check off tasks on the checklist when completed, to assist them in working through the tasks. The first alarm occurred 30 min before the first saliva sample, at which time the checklist prompted participants to close all blinds and curtains in their home environment, to reduce exposure to any outdoor light, and to turn off or dim indoor lights (including bathroom lights) to the lowest level possible while still permitting the reading of the checklist. Participants were also instructed to dim the screens of electronic devices they anticipated using during the home phase assessment, including televisions, computers, cell phones, and music-playing devices. The light from the timer and night light were dim (˜1.5 lux and 3.5 lux respectively, at level of eyes, in direction of gaze, measured ˜42 cm from eye, Extech 403125 light meter). The checklist also prompted participants to place the test tube rack and removable ice pack in their freezer, and to use the attached safety pin to pin the light medallion to their outer most clothing. All other pieces of the home kit were to be placed on a nearby table for easy access. As in the laboratory phase assessments, small snacks and fluids were permitted, except in the 10 min before each sample, when participants were prompted by the alarm/checklist to brush their teeth with the toothbrush if they had eaten any food, to rinse with water if they had consumed anything apart from water, and to remain seated until after the next saliva sample. Compliance to this instruction was not assessed. At each scheduled time for a saliva sample, the alarms/checklist prompted participants to open the Track Cap lid (which recorded time of opening), remove a cotton swab from the vial, replace the Track Cap lid, roll the cotton swab in their mouths for up to 5 min until saturated, spit the cotton swab into an empty Salivette, attach a label from the label dispenser to the Salivette, and place the Salivette in the test tube rack in their freezer. As in the laboratory phase assessments, showers and exercise, toothpaste or mouthwash was not allowed during the home phase assessments. The checklist also contained the telephone number of a staff member to call if any questions came up during the home phase assessment, although only two participants called the number, with questions about the timer. After the last saliva sample was obtained, the checklist prompted participants to remove the light medallion and place it face up on their bedside table, turn off all lights, and to go to bed to sleep. The following morning, at the participants' average wake time, the checklist prompted participants to put the light medallion back on. When participants were ready to return to the laboratory, the checklist instructed them to place the ice pack and frozen Salivettes in the small insulated bag, and to pack all remaining equipment into the larger messenger bag.

Preliminary Data Analysis

When participants returned to the laboratory to drop off the home kit, the research staff checked that all contents of the kit were returned, and participants were asked how many people were home during the home phase assessment and their respective ages. The number of people home during the home phase assessment ranged from zero to six people, and at least one other person was present for the majority of the home phase assessments (64%). The age of the people present during the home phase assessments ranged from 6 to 84 y. The light medallion was removed from the subject and downloaded, and the activity on the light medallion was checked to confirm participants were wearing the light medallion as instructed. The light levels from the 30 min before the first saliva sample to the last saliva sample were also checked, with light levels <50 lux coded as compliant and light levels 50 lux coded as non-compliant. This light threshold was chosen based on an illuminance response curve generated in dark adapted participants, which indicates minimal melatonin suppression at light intensities <50 lix.²⁶ The Track Cap was also downloaded (Power-View version 3.4.1, MWV Healthcare, Richmond, Va.), with any saliva samples 5 min from the scheduled time coded as compliant, and samples taken >5 min from the scheduled time coded as noncompliant.

The Salivettes collected in the laboratory were immediately centrifuged to extract the saliva from the cotton swab and then frozen. The Salivettes collected at home were thawed, centrifuged, and then refrozen. The saliva samples were then shipped in dry ice to Solidphase Inc. (Portland, Me.) which radioimmunoassayed the samples for melatonin using commercially available kits (ALPCO, Inc, Salem, N.H.). Each individual's saliva samples were assayed in the same batch. The first non-zero standard of this assay was 0.5 pg/mL. Intra-assay coefficients of variation for low, medium, and high levels of salivary melatonin are 20.1%, 4.1%, and 4.8%, respectively. The interassay coefficients of variation for low, medium, and high levels of salivary melatonin are 16.7%, 6.6%, and 8.4%, respectively. A DLMO was calculated for each phase assessment and defined as the clock time (with linear interpolation) when the melatonin concentration exceeded the mean of three low consecutive daytime values plus twice the standard deviation of these points.²⁷ This low threshold more closely tracks the initial rise of melatonin.²⁸

Results

Compliance to the Scheduled Bed and Wake Times

The wrist activity revealed that all participants except two demonstrated good compliance to the study protocol, going to bed and getting out of bed at home within 15 min of the assigned times. The two noncompliant participants each slept up to 1.5 h after their assigned wake time after their first home phase assessment, but before the first laboratory phase assessment in Protocol A.

Compliance to the Requirement for Dim Light

Each of the participants completed a home phase assessment twice. Thirteen participants (41% of the sample) received at least one 30-sec epoch of light >50 lux during both 8.5 h home phase assessments, eleven participants (34%) received such light during only one of the two home phase assessments, whereas eight participants (25%) were able to remain in dim light throughout both home phase assessments. Overall, the median frequency of 30-sec epochs>50 lux was 2. The average difference in number of epochs>50 lux between the two home phase assessments was 15.7±34.7. Often the light>50 lux was received in the first 30 min of the home phase assessment (58% of the time), as participants began to close their blinds and curtains and dimmed their inside lighting. When light>50 lux did occur during the 8.5 h home phase assessment, the duration lasted between 30 sec to 95 min, and on average lasted for 8.8±16.3 min (or on average 1.7% of the home phase assessment). The average light intensity during home phase assessments was 4.5 lux, with a range of zero to 13,047 lux (the maximum occurring in the first 2 min of the home phase assessment as the subject closed her blinds). The average light intensity of the epochs with light>50 lux was 158.5 lux. The most common activity during the home phase assessments was watching television (63%), followed by reading (19%), using a computer (9%), and housework (9%). There was no significant relationship between the occurrence of light>50 lux during the home phase assessments and subject characteristics such as age, sex, race, education, employment status, student status, number of people home, youngest age of people home, oldest age of people home, and whether the first sample was before or after sunset (all P>0.12). There was a trend for more participants in Protocol A (81%) than in Protocol B (50%) to receive light>50 lux in the first home phase assessment (chi-square, P=0.063), but not in the second home phase assessment (chi-square, P=0.48). This is most likely because in the first home phase assessment participants in Protocol A had not yet experienced the dim light in a laboratory phase assessment.

Compliance to Scheduled Sample Times

Three participants (9% of the sample) collected at least one saliva sample more than 5 min from a scheduled sample time in both home phase assessments, 11 participants (34%) collected at least one saliva sample more than 5 min from a scheduled sample time in only one home phase assessment, and 18 participants (56%) had no problem collecting saliva samples within 5 min of the scheduled times in both home phase assessments. Two subjects missed one sample during their first home phase assessment. The majority of sample errors resulted in samples collected within 11 min of the scheduled time (88% of the errors), and mostly occurred when participants mistakenly collected a saliva sample when the timer/checklist prompted them to brush their teeth and rinse with water in the 10 min before a scheduled sample. There was no significant relationship between compliance to the scheduled sample times during the home phase assessments and subject characteristics such as age, sex, race, education, employment status, student status, number of people home, youngest age of people home, oldest age of people home, or whether the subject participated in Protocol A or B (all P≧0.15).

Dim Light Melatonin Onsets

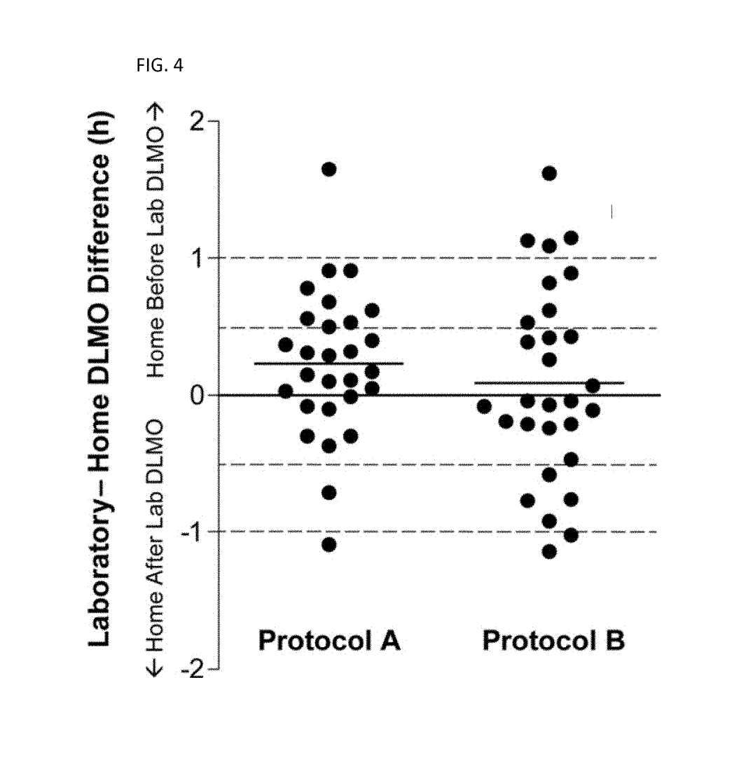

One subject who ran in Protocol A consistently secreted a low level of melatonin (<5 pg/mL) and there was no discernible onset in melatonin secretion in all home and laboratory phase assessments. Thus, there were no DLMOs from this subject. After all of the home DLMOs were calculated, they were cross checked against the light levels on the light medallion and sample times from the TrackCap. If a sampling error affected one of the two melatonin data points below and above the threshold, which are used in the calculation of the DLMO, the home DLMO was considered invalid. This occurred on two occasions. Similarly, if light exposure was >50 lux within 30 min of the two melatonin data points used in the calculation of the DLMO, the home DLMO was considered likely suppressed and invalid. This occurred on three occasions. This rule was derived from data showing melatonin rebounded in ˜30 min after a 12 min exposure to very bright light (10,000 lux, FIG. 4 in Chang et Thus, of the 62 home DLMOs calculated, 57 (92%) were considered valid.

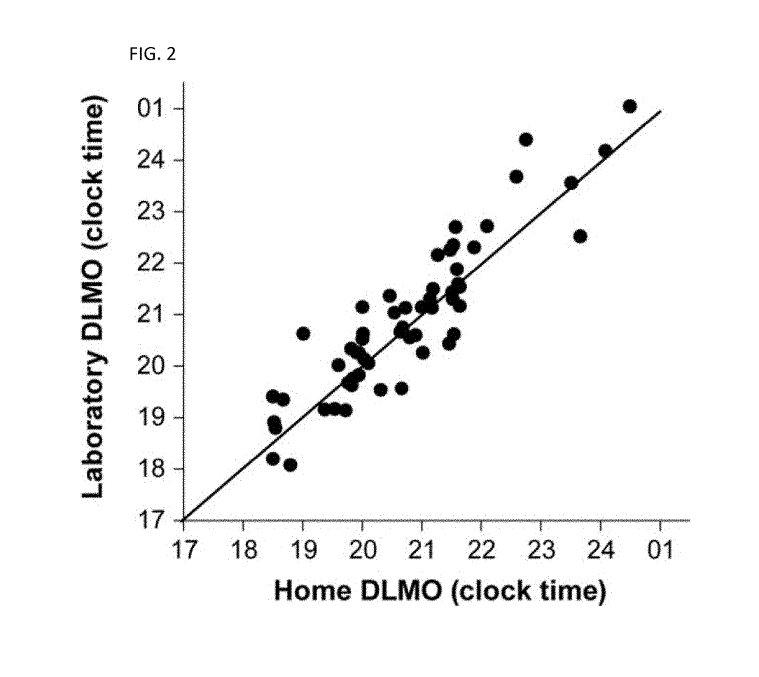

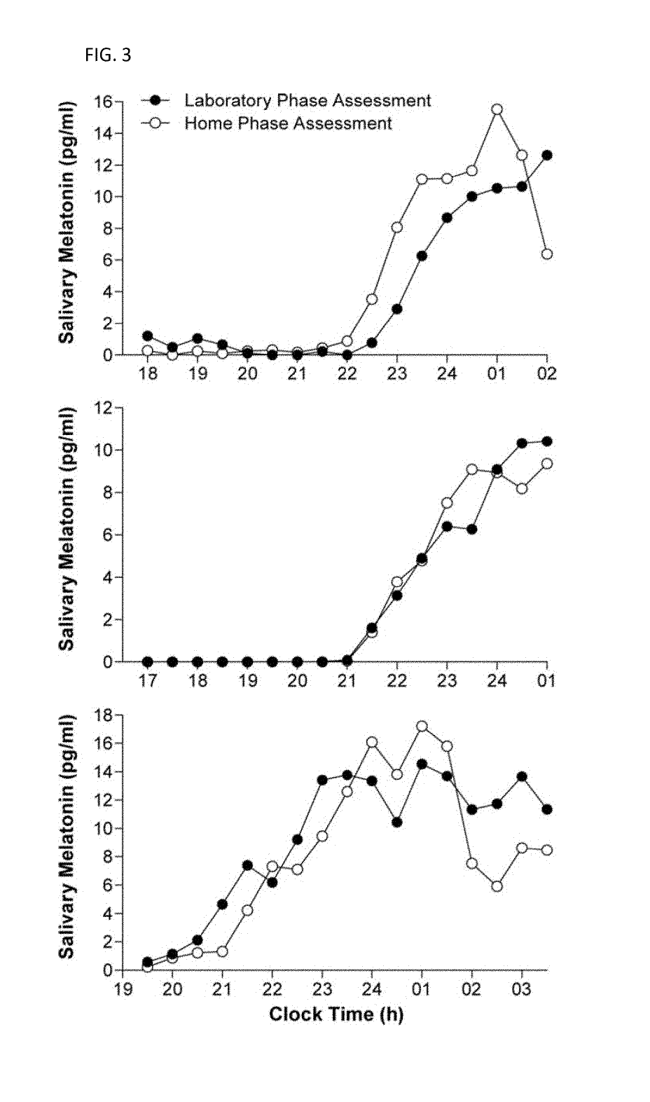

The home and laboratory DLMOs were highly correlated (r=0.91, P<0.001) (FIG. 2). Individual examples of when the home DLMO occurred more than 30 min before, at approximately the same time, or more than 30 min after the laboratory DLMOs are shown in FIG. 3. Each valid home DLMO was subtracted from the laboratory DLMO that occurred immediately before or after that home DLMO. Thus a positive number indicated the home DLMO occurred before the laboratory DLMO, whereas a negative number indicated the home DLMO occurred after the laboratory DLMO. Overall the average difference between the home and laboratory DLMO in each pair of DLMOs was 0.16±0.63 h, reflecting that on average the home DLMO occurred before the laboratory DLMO. However, there was no significant difference between the home and laboratory DLMOs (paired t test, P>0.05). The distribution of the differences between each pair of home and laboratory DLMOs was normally distributed (skew=0.07±0.32, kurtosis=−0.01±0.62, FIG. 4). In 33 cases (58% of the data) the home DLMO occurred within 30 min of the laboratory DLMO. In 16 cases (28% of the data) the home DLMO occurred more than 30 min earlier than the laboratory DLMO (maximum difference 1.65 h earlier). In eight cases (14% of the data), the home DLMO occurred more than 30 min after the laboratory DLMO (maximum difference 1.14 h later). In both protocols it was less common for the home DLMO to occur after the laboratory DLMO (as might be expected if melatonin suppression was occurring during the home phase assessments). Similarly, the magnitude of the difference between the home and laboratory DLMOs was always less when the home DLMO occurred after the laboratory DLMO. Thus in sum, there was no evidence that the home phase assessments systematically led to later DLMOs than those measured in the laboratory. In 58% of cases the home DLMO occurred within 30 min of the laboratory DLMO and in 88% of cases the home DLMO occurred within 1 h of the laboratory DLMO. The average difference between the two home DLMOs was 0.36±0.68 h and the average difference between the two laboratory DLMOs was 0.48±0.86 h. The average difference between the two home DLMOs and the average difference in number of epochs>50 lux between the two home phase assessments were not significantly correlated (r=0.19, P=0.33).

Discussion

This study is the first test of a novel kit designed to facilitate home saliva sampling for later determination of the DLMO. The home procedure included objective measures of compliance to the requirement for dim light and scheduled times for saliva samples, and a system to reduce labeling errors. Overall participants were reasonably compliant to the requirement for dim light. Although 75% of the participants received at least one 30-sec epoch of light>50 lux during their home phase assessments, the average duration of light>50 lux in these participants was less than 9 min of the required 8.5 h of dim light. Participants were also reasonably compliant to the requirement for saliva samples every half hour, with more than half of the participants collecting all their home saliva samples within 5 min of the scheduled times. Thus overall, compliance to the home procedures was good and the light data from the photo-sensor and sample timing data from the medication monitoring device indicated 92% of the home DLMOs were valid with these relatively strict criteria.

The home DLMOs correlated highly with the laboratory based DLMOs (r=0.91). This correlation is considerably higher than the correlation between home and laboratory DLMOs previously observed in a study with no measures of light exposure or sample timing (r=0.68).¹² Importantly, the previous study and the current study used the same low threshold to calculate the DLMOs,^{11,27} and so the DLMOs are directly comparable. In this earlier study, 20% of home DLMOs occurred 1 h or more after the corresponding laboratory DLMOs (suggesting light-induced melatonin suppression at home),¹² whereas only 4% of home DLMOs in the current sample occurred 1 h or more after the corresponding laboratory DLMO. Furthermore, the average difference between home and laboratory DLMOs was less than 10 min in the current study versus the previously observed 54 min,¹² and less than the 30-min sampling rate. Indeed, the maximum difference between the home and laboratory DLMOs occurred when a home DLMO occurred 1.65 h earlier in time than the corresponding laboratory DLMO. This difference falls within the 95% confidence intervals surrounding the mean difference between two laboratory DLMOs assessed about 3 w apart in healthy participants sleeping on a fixed sleep schedule (±30 min).³⁰ In that study, the upper limit of the 95% confidence interval was a difference of 2.4 h between the two laboratory DLMOs.³⁰ Similarly, the weekly difference falls within the difference observed between two laboratory DLMOs assessed at least 5 days apart in healthy participants sleeping on an ad lib schedule³¹ (FIG. 2). The observed variability in the difference between the home and laboratory DLMOs in the current study is most likely due to the typical variations in the sleep times of the participants before each back-to-back phase assessment By contrast, the 2 h of dim light after habitual bedtime²⁶ and the 2 h afternoon nap before the second phase assessments³⁴ are unlikely to have significantly shifted the DLMO. Overall, the good agreement between the home and laboratory DLMOs in this study suggests that including objective measures of light exposure and sample timing during home saliva sampling, and also informing participants that their compliance is being monitored, can lead to more accurate home DLMOs.

The home saliva sampling procedure tested here is the next step toward developing a standardized approach to measure valid DLMOs at home. Home DLMOs offer several advantages over laboratory or clinic based DLMOs, including reduced cost, and potentially greater accessibility to patient groups that are reluctant to stay overnight in a facility (e.g., postpartum women). The home procedures used in this study required participants to give half-hourly saliva samples, which is significant considering other protocols for home saliva sampling have relied on only hourly sampling.^{12,14} Half-hourly sampling at home was required as this higher sampling frequency is often used in the laboratory or clinic, and thus provided the optimal comparison to laboratory DLMOs.²⁸ Nonetheless, hourly sampling may be more practical for clinical practice.²⁸ The saliva collection window was tailored to each subject, starting 6 h before each subject's average bedtime and continuing up to 2 h after each subject's average bedtime. A similar 8-h sampling window was used previously, although it was shifted 1 h earlier, with saliva sampling starting 7 h before and ending 1 h after habitual bedtime.¹² Other home saliva sampling protocols have used only a 5-h sampling window.¹⁴ In the current study the earliest DLMO relative to sampling time occurred 2 h after the first saliva sample and the latest DLMO occurred 1 h before the last sample, suggesting the full 8-h window may be needed to best capture the DLMO, at least in healthy people.

Although the kit and procedures worked quite well, there are several areas for potential improvement in future studies. One possibility is the addition of “blue blocker” glasses, which can minimize any melatonin suppression due to indoor lighting.^{35,36} Such glasses were not included in the kit tested here, because of the difficulty in measuring subject compliance in wearing them. Nonetheless, participants could be encouraged to wear them during home phase assessments, as in a previous study participants reported wearing them about 70% of the requested time.³⁷ Another change could be to use a small personal electronic device such as a smart phone, as the timer in the kit instead of a personal data assistant (PDA), as the PDA was somewhat burdensome to staff, requiring careful programming of the alarms with specialized desktop software. Additionally, the staff time taken to explain the kit to each and every subject could conceivably be replaced with a short video explaining the procedure, followed by a short question and answer session with staff. Participants were also less likely to receive light>50 lux during the home phase assessments, after they had experienced the laboratory phase assessment (<5 lux). Thus, people may be more successful at ensuring dim light at home if they are first shown a room with appropriate dim lighting, as verified by a staff member with a light meter. Finally, participants may experience less sampling errors if they are informed that the foremost sampling error was to generate a saliva sample about 10 min early, when instead they should have been brushing their teeth to remove food particles.

The home saliva sampling procedure in this study was tested in healthy participants whose age ranged between 21-62 y. Interestingly, subject compliance to the requirement for dim light and sample times were not significantly associated with any characteristics of the participants such as their age, race, education, employment status or student status. Similarly, there were no significant relationships between characteristics of the participants' home environment and their compliance, such as the number of people home during the home phase assessment and their respective ages. One subject successfully completed both home phase assessments with six other family members present and the difference between his home and laboratory DLMOs was less than 5 min on both occasions. Nonetheless, the sample was highly educated, with 94% of subjects having started some college education, indicating further validation of the procedure may be required in a more representative sample of the general population. Nonetheless, all the participants were healthy, and the home saliva sampling kit and procedure remains to be tested in patient populations, including those with extremes of chronotype. Given the greater variability in the sleep-wake schedules of patients with various circadian rhythm sleep disorders, including delayed sleep-wake phase disorder, there is likely to be larger variability between home-and laboratory-based phase assessments. The home kit and procedures will also need validation for use in children and older adults with neurodegenerative conditions, as both will require assistance with the home procedures. As a next step we are testing the home saliva sampling procedure in patients with delayed sleep-wake phase disorder because the DLMO can be quite useful in the differential diagnosis of this disorder.³⁸

### Example 2

### HDLOs with Measures of Compliance in Delayed Sleep Phase Disorder

Methods

Thirty-seven patients participated year-round, days from daylight savings time changes. All were medication free, with body mass index (BMI) 17.0-33.7 kg/m². Based on screening (Burgess et al., 2015), all patients had no medical disorders. Each participant was interviewed by a board-certified sleep clinician (MP, JW) who confirmed that they met ICSD-2 criteria for DSPD and were low risk for seasonal affective disorder. Five participants tested positive for nicotine or benzodiazepines at study start and were not included in the analyses. The remaining 32 participants included 17 women; mean age±standard deviation (SD): 26.3±6.5 years. There were five neither, 20 moderate evening and seven definite evening types (Home and Ostberg, 1976). None had worked any night shifts nor travelled across more than one time zone in the previous month. Non-steroidal anti-inflammatory drugs were not permitted. Participants gave written informed consent prior to participation. The study was approved by Rush University Medical Center Institutional Review Board.

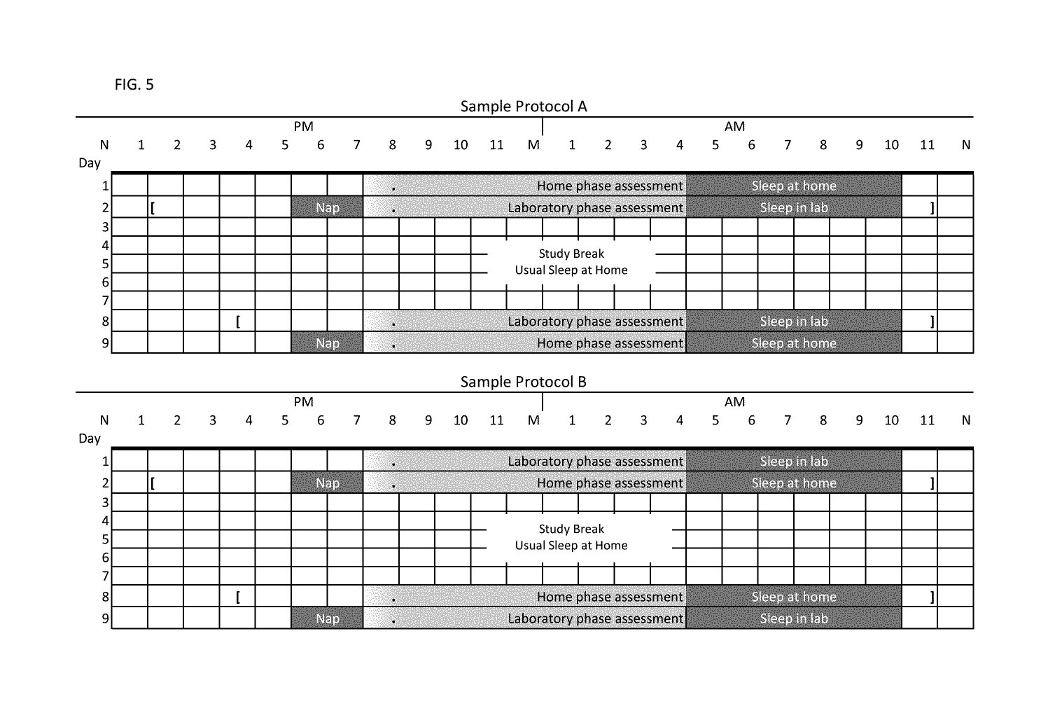

The protocol was tailored to each individual's habitual sleep times collected during the week before study start (FIG. 5). Each individual participated in two back-to-back laboratory and home DLMO assessments, starting saliva collection 6 h before habitual sleep onset (Burgess et al., 2015). Thus, each participant generated two home-laboratory DLMO pairs. Participants wore a wrist actigraph (Actiwatch Spectrum; Philips Healthcare, Andover, Mass., USA) to ensure compliance. Light levels 50 lux from 30 min before the first saliva sample to the last sample were considered non-compliant. A vial with a MEMS® TrackCap lid (MVW Healthcare, Richmond, Va., USA) was downloaded, with any samples taken >5 min from the scheduled time considered non-compliant. All saliva samples were frozen at −20° C. after centrifuging and radioimmunoassayed (ALPCO, Inc., Macedon, N.Y., USA). DLMOs were calculated as the time when melatonin levels exceeded the mean of three low consecutive values (before rise) plus twice the standard deviation (Molina and Burgess, 2011). Only two points could be used for three participants due to their early melatonin profiles.

Results

Nineteen participants completed protocol A and 13 completed protocol B. The average sleep onset and wake times were 02:20±1.1 hours (00:00-04:30 hours) and 10:00±1.5 hours (07:00-12:30 hours), and 19% of participants habitually slept 6.5 h night_1.

Seven participants (22%) received at least one epoch of light>50 lux during both home assessments, 14 (44%) during only one home assessment, while 10 (31%) remained in dim light throughout both home assessments. Often, the light>50 lux occurred during the first 30 min (43%), as blinds/curtains were closed and lighting dimmed. When light>50 lux occurred, the duration lasted between 30 s and 36.5 min (median 3.0 min, 1.5% of time). The median light intensity was 0.8 lux (0-50 937 lux).

Three participants (9%) missed between one and three samples. Four participants (13%) collected at least one sample>5 min from a scheduled time in both home assessments, seven (22%) during only one home assessment and 18 (56%) collected all samples on time.

One participant displayed unusually early melatonin profiles, and the DLMO was missed in all assessments. In a second participant, this occurred in the first two assessments only. One participant had blood in her saliva in one home assessment, and in another the light medallion failed. Thus, 59 home-laboratory DLMO pairs were analyzed. The home DLMOs were cross-checked against light levels and recorded sample times. On five occasions, a sampling error affected the data points used to calculate the DLMO, and the DLMOs were considered invalid. On five occasions, light>50 lux occurred within 30 min of the data-points, and the DLMOs were considered invalid. Thus, of the 59 home DLMOs, 49 (83%) were considered valid. In a non-significant trend, the invalid DLMOs occurred on average 0.91 h later than the laboratory DLMOs (P=0.073).

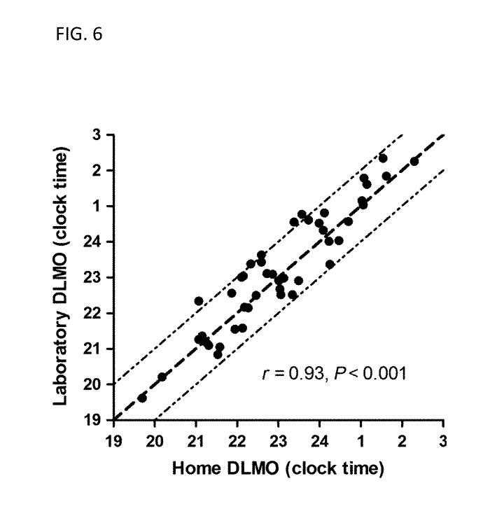

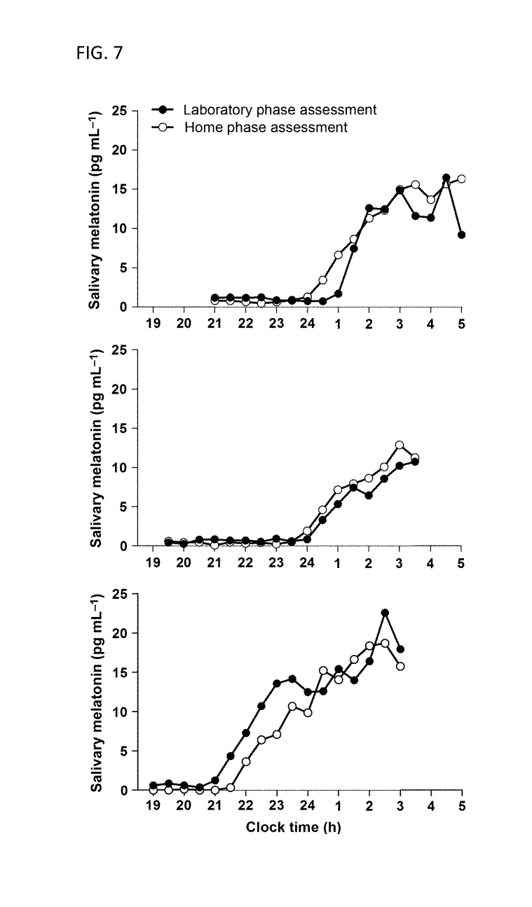

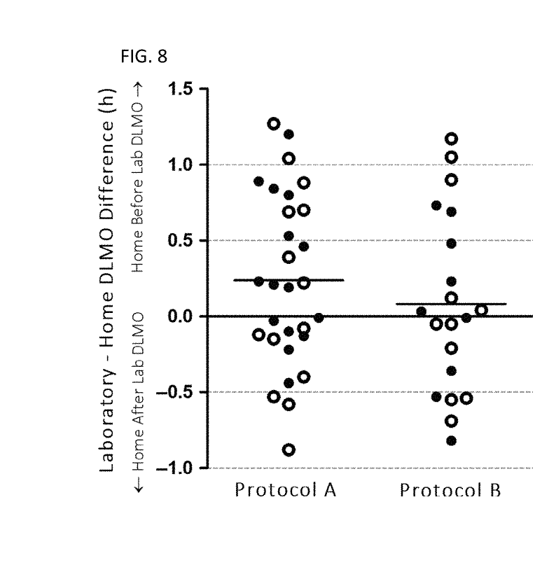

The home and laboratory DLMOs were correlated highly (FIG. 6). FIG. 7 shows examples of laboratory-home DLMO pairs. Each valid home DLMO was subtracted from its adjacent laboratory DLMO. In 10% of cases the home DLMO was more than 1 h from the laboratory DLMO. The average difference in each pair was 0.17±0.58 h, reflecting that on average the home DLMO occurred before the laboratory DLMO (P=0.041, FIG. 8). It was less common for the home DLMO to occur after the laboratory DLMO. Similarly, the difference between the home and laboratory DLMOs was always less when the home DLMO occurred after the laboratory DLMO. Thus, there was no evidence that the home assessments led to later DLMOs.

Discussion

This is the first test of our kit and procedures in patients with a circadian rhythm sleep disorder. Compliance with the requirement for dim light and half-hourly saliva sampling was similar to controls (Burgess et al., 2015). However, light and sample timing errors were more frequent near the DLMO, leading to more invalid home DLMOs in the DPSDs (17% versus 8% in controls). The high level of compliance is due probably to participants knowing that their compliance was being monitored (Kudielka et al., 2003).

The valid home DLMOs occurred, on average, only 10 min before the laboratory DLMOs, and they were correlated highly. The maximum difference occurred when a home DLMO was 1.27 h earlier than the laboratory DLMO. This is less than the 2-h difference observed between two laboratory DLMOs assessed days apart in DSPDs who slept ad libitum (Wyatt et al., 2006). The observed variability is due most probably to the ˜2.5- to 3-h variation in sleep times before each back-to-back assessment. The good agreement between the DLMOs suggests that home DLMOs collected with measures of compliance compare favourably to laboratory DLMOs in DPSD. The home DLMOs were not systematically later than the laboratory DLMOs, which would be expected if melatonin suppression occurred at home.

The home procedures tested here are the next step towards developing a standardized approach to measure DLMOs at home. The saliva collection started 6 h before each participant's average self-reported sleep onset, but several suspected early DLMOs were missed. However, all DLMOs occurred>3 h before the end of saliva sampling, suggesting that DSPDs may not need to provide samples beyond their usual sleep onset time. We continue to improve our procedures, now demonstrating dim light to participants and using an iPod touch instead of a personal digital assistant (PDA). This work demonstrates that home saliva sampling with measures of compliance is feasible, and can help to distinguish between valid home DLMOs and DLMOs affected by light or sampling errors.

It is therefore intended that the foregoing detailed description be regarded as illustrative rather than limiting, and that it be understood that it is the following claims, including all equivalents, that are intended to define the spirit and scope of this invention.

### REFERENCES

1. Lewy A J, Cutler N L, Sack R L. The endogenous melatonin profile as a marker of circadian phase position. J Biol Rhythms. 1999;14:227-36.

2. Klerman E B, Gershengorn H B, Duffy J F, Kronauer R E. Comparisons of the variability of three markers of the human circadian pacemaker. J Biol Rhythms. 2002;17:181-93.

3. Burgess H J, Fogg L F. Individual differences in the amount and timing of salivary melatonin secretion. PLoS One. 2008;3:e3055.

4. Lewy A J, Wehr T A, Goodwin F K, Newsome D A, Markey S P. Light suppresses melatonin secretion in humans. Science. 1980;210:1267-9.

5. American Academy of Sleep Medicine. The International Classification of Sleep Disorders. 3rd Edition. Darien, Ill.: American Academy of Sleep Medicine; 2014.

6. Lockley S W. Timed melatonin treatment for delayed sleep phase syndrome: the importance of knowing circadian phase. Sleep. 2005;28:1214-6.

7. Keijzer H, Smits M G, Duffy J F, Curfs L M. Why the dim light melatonin onset (DLMO) should be measured before treatment of patients with circadian rhythm sleep disorders. Sleep Med Rev. 2014;18:333-9.

8. Strollo P J, Jr., Badr M S, Coppola M P, Fleishman S A, Jacobowitz O, Kushida C A. The future of sleep medicine. Sleep. 2011;34:1613-9.

9. Bjorvatn B, Pallesen S. A practical approach to circadian rhythm sleep disorders. Sleep Med Rev. 2009;13:47-60.

10. Lewy A J, Lefler B J, Emens J S, Bauer V K. The circadian basis of winter depression. Proc Natl Acad Sci U S A. 2006;103:7414-9.

11. Benloucif S, Burgess H J, Klerman E B, et al. Measuring melatonin in humans. J Clin Sleep Med. 2008;4:66-9.

12. Pullman R E, Roepke S E, Duffy J F. Laboratory validation of an in-home method for assessing circadian phase using dim light melatonin onset (DLMO) Sleep Med. 2012;13:703-6.

13. Kudielka B M, Broderick J E, Kirschbaum C. Compliance with saliva sampling protocols: electronic monitoring reveals invalid cortisol daytime profiles in noncompliant subjects. Psychosom Med. 2003;65:313-9.

14. Keijzer H, Smits M G, Peeters T, Looman C W, Endenburg S C, Gunnewiek J M. Evaluation of salivary melatonin measurements for Dim Light Melatonin Onset calculations in patients with possible sleep-wake rhythm disorders. Clin Chim Acta. 2011;412:1616-20.

15. Tasto D L, Colligan M J, Skjei E W, Polly S J. Health consequences of shift work. Cincinnati, Ohio.: NIOSH Publication #78-154; 1978.

16. Butcher J N, Dahlstrom W G, Graham J R, Tellegen A, Kaemmer B. Minneapolis, Minn.: University of Minnesota Press; 1989. MMPI-2 (Minnesota Multiphasic Personality Inventory-2): manual for administration and scoring.

17. Beck A T, Ward C H, Mendelson M, Mock J, Erbaugh J. An inventory for measuring depression. Arch General Psychiatry. 1961;4:53-63.

18. Terman M, Williams J. Personal inventory for depression and SAD (PIDS) J Prac Psychiatry Behav Health. 1998;5:301-3.

19. Buysse D J, Reynolds C F, Monk T H, Berman S R, Kupfer D J. The Pittsburgh sleep quality index: a new instrument for psychiatric practice and research. Psychiatry Res. 1989;28:193-213.

20. Bastien C H, Vallieres A, Morin C M. Validation of the Insomnia Severity Index as an outcome measure for insomnia research. Sleep Med. 2001;2:297-307.

21. Netzer N C, Stoohs R A, Netzer C M, Clark K, Strohl K P. Using the Berlin Questionnaire to identify patients at risk for the sleep apnea syndrome. Ann Intern Med. 1999;131:485-91.

22. Allen R P, Picchietti D L, Garcia-Borreguero D, et al. Restless legs syndrome/Willis-Ekbom disease diagnostic criteria: updated International Restless Legs Syndrome Study Group (IRLSSG) consensus criteria—history, rationale, description, and significance. Sleep Med. 2014;15:860-73.

23. Horne J A, Ostberg O. A self-assessment questionnaire to determine morningness-eveningness in human circadian rhythms. Int J Chronobiol. 1976;4:97-110.

24. Murphy P J, Myers B L, Badia P. Nonsteroidal anti-inflammatory drugs alter body temperature and suppress melatonin in humans. Physiol Behay. 1996;59:133-9.

25. Burgess H J. Evening ambient light exposure can reduce circadian phase advances to morning light independent of sleep deprivation. J Sleep Res. 2013;22:83-8.

26. Zeitzer J M, Dijk D J, Kronauer R E, Brown E N, Czeisler C A. Sensitivity of the human circadian pacemaker to nocturnal light: melatonin phase resetting and suppression. J Physiol. 2000;526.3:695-702.

27. Voultsios A, Kennaway D J, Dawson D. Salivary melatonin as a circadian phase marker: validation and comparison to plasma melatonin. J Biol Rhythms. 1997;12:457-66.

28. Molina T A, Burgess H J. Calculating the dim light melatonin onset: the impact of threshold and sampling rate. Chronobiol Int. 2011;28:714-8.

29. Chang A M, Santhi N, St Hilaire M A, et al. Human responses to bright light of different durations. J Physiol. 2012;590:3103-12.

30. Benloucif S, Guico M J, Reid K J, Wolfe L F, L'Hermite-Baleriaux M, Zee P C. Stability of melatonin and temperature as circadian phase markers and their relation to sleep times in humans. J Biol Rhythms. 2005;20:178-88.

31. Wyatt J K, Stepanski E J, Kirkby J. Circadian phase in delayed sleep phase syndrome: predictors and temporal stability across multiple assessments. Sleep. 2006;29:1075-80.

32. Burgess H J, Eastman C I. Early versus late bedtimes phase shift the human dim light melatonin rhythm despite a fixed morning lights on time. Neurosci Lett. 2004;356:115-8.

33. Burgess H J, Eastman C I. A late wake time phase delays the human dim light melatonin rhythm. Neurosci Lett. 2006;395:191-5.

34. Buxton O M, L'Hermite-Baleriaux M, Turek F W, Van Cauter E. Daytime naps in darkness phase shift the human circadian rhythms of melatonin and thyrotropin secretion. Am J Physiol. 2000;278:R373-82.

35. Sasseville A, Paquet N, Sevigny J, Hebert M. Blue blocker glasses impede the capacity of bright light to suppress melatonin production. J Pineal Res. 2006;41:73-8.

36. Kayumov L, Casper R F, Hawa R J, et al. Blocking low-wavelength light prevents nocturnal melatonin suppression with no adverse effect on performance during simulated shift work. J Clin Endocrinol Metab. 2005;90:2755-61.

37. Burgess H J, Molina T A. Home lighting before usual bedtime impacts circadian timing: a field study. Photochem Photobiol. 2014;90:723-6.

38. Rahman S A, Kayumov L, Tchmoutina E A, Shapiro C M. Clinical efficacy of dim light melatonin onset testing in diagnosing delayed sleep phase syndrome. Sleep Med. 2009;10:549-55.

39. Burgess, H. J., Wyatt, J. K., Park, M. and Fogg, L. F. Home circadian phase assessments with measures of compliance yield accurate dim light melatonin onsets. Sleep, 2015, 38: 889-897.

1. A method of collecting samples for assessment of circadian timing, the method comprising:
monitoring light exposure of a subject in a non-clinical environment;
placing a first biological sample from a subject in a first sample vial, wherein the first biological sample is obtained in the non-clinical environment in dim light comprising light less than about 50 lux;
monitoring the use of the first sample vial with a monitoring device;
placing a second biological sample from the subject in a second sample vial, the second biological sample being taking at a time interval after the first biological sample; and
monitoring the use of the second sample vial with the monitoring device.

2. The method according to claim 1, further comprising labeling the first sample vial with a first pre-coded label, wherein the first pre-coded label is added to the first sample vial in the non-clinical environment.

3. The method according to claim 1, comprising alerting the subject to a first sampling time for the first biological sample using a timer.

4. The method according to claim 1, comprising providing a checklist with instructions for the subject.

5. The method according to claim 1, comprising monitoring sampling times using the monitoring device.

6. The method according to claim 1, comprising obtaining biological samples at about 30 minute intervals.

7. The method according to claim 1, wherein the biological sample comprises saliva.

8. The method according to claim 1, wherein the light exposure is monitored prior to the first sample being placed in the first sample vial through a final sample being placed in a final sample vial.

9. The method according to claim 1, comprising providing a plurality of pre-coded labels that are dispensed in order so that a first pre-coded label is applied to the first sample vial and a second pre-coded label is applied to the second sample vial.

10. The method according to claim 1, comprising positioning a photosensor on the subject so the photosensor is free from covering for monitoring light exposure.

11. The method according to claim 1, comprising monitoring the light exposure in 30-sec epochs.

12. A kit for collecting samples for assessment of circadian timing, the kit comprising:
a photosensor;
a plurality of sample vials free from labels;
a label system for labeling the plurality of sample vials; and
a monitoring device for monitoring use of the plurality of sample vials.

13. The kit according to claim 12, further comprising a timer.

14. The kit according to claim 13, wherein the timer comprises voice prompts.

15. The kit according to claim 12, wherein the monitoring device comprises a track cap for registering a sample time with the monitoring device.

16. The kit according to claim 12, comprising a check list to advise a subject of sampling times.

17. The kit according to claim 12, wherein the photosensor measures light exposure in 30-sec epochs.

18. The kit according to claim 12, further comprising a plurality of swabs.

19. The kit according to claim 12, comprising a sample vial holder.

20. The kit according to claim 12, comprising a night light, wherein light from the night light is less than 50 lux.
